(19) United States
(12) Patent Application Publication
(10) Pub. No.: US 2006/0100463 A1
(43) Pub. Date: May 11, 2006

(54) Selective hydrogenation of nitrogen containing aromatics

(76) Inventors: Avinash Narendra Mahajan, Maharashtra (IN); Prakash Shivanand Ozarde, Maharashtra (IN); Raj Madhukar Deshpande, Maharashtra (IN); Raghunath Vitthal Chaudhari, Maharashtra (IN)

Correspondence Address: John P White;Cooper & Dunham, 1185 Avenue of the Americas, New York, NY, 10036, US

(21) Appl. No.: 10/490,757

(22) PCT Filed: Dec. 24, 2003
(86) PCT No.: PCT/IB03/06185

§ 371 Date: Jan. 3, 2006

(51) Int. Cl. C07C 209/38; C07C 209/36

(52) U.S. Cl. 564/422

(57) ABSTRACT

The present invention describes an improved catalyst and process for the selective hydrogenation of nitro aromatics, nitrosoaromatics and aromatic hydroxylamines to their corresponding amines using gaseous hydrogen, in the presence of soluble iron compounds as a catalyst and optionally other reducible groups.

### TECHNICAL FIELD

The present invention relates to an improved process for the selective hydrogenation of nitro aromatics. The homogeneous iron catalysts of the present invention catalyze the selective hydrogenation of substituted and unsubstituted nitro aromatics, nitrosoaromatics and aromatic hydroxylamines to aromatic amines with gaseous hydrogen in the presence of other substituent functional groups that are reducible or susceptible to hydrogenolysis.

### BACKGROUND AND THE PRIOR ART OF THE INVENTION

A large variety of aromatic amines are widely used for the production of polymers, dyes, pharmaceuticals, agrochemicals and photographic chemicals (P. F. Vogt, J. J. Gerulis, ‘Aromatic Amines’ in Ullmann's Encyclopedia, 5^{th} Ed Vol A2, Verlag Chemie, Weinheim (1885) 37-55). In earlier days the reduction of nitro aromatics using iron/acid discovered by Bechamp (Bechamp A. J., Ann. Chim. Phys, 1854, [3], 42, 186), was extensively used, though, nowadays, this process has lost relevance for products such as aniline due to availability of efficient heterogeneous catalysts. The Bechamp process, despite being corrosive and polluting still remains a method of choice for reduction of specialty nitro aromatics due to its remarkable selectivity against other reducible functional groups. References may be made to U. Siegrist, P. Baumeister, H. U. Blaser, M. Studer, Chem. Ind. (Dekker 75 (1998) 207, and Blaser H. U. and Studer M., Appl. Catal. 1999, Vol 189, pages 191-204, wherein the controlled poisoning of heterogeneous noble metal catalyst has also been used to achieve selective hydrogenation of nitro functional group as described. Although these catalysts selectively reduce nitro aromatics they suffer from a drawback, which is that the catalyst preparation process often remains critical and amount of poison needs to be precisely controlled.

In U.S. Pat. No. 3,832,401 (1974), the hydrogenation of nitroaromatics has been claimed using the specific Fe complexes Fe(CO)₃(PPh₃)₂, Fe(acac)₃ and Fe(CO)₃(AsPh₃)₂, however the advantage of the present invention over U.S. Pat. No. 3,832,401 is that the catalysts and process employing these can be carried out in organic, aqueous and combination of organic-aqueous media. In contrast it is specifically mentioned in U.S. Pat. No. 3,832,401 that non-aqueous solvent medium is necessary for reaction.

The use of iron doped Raney Nickel catalysts for the hydrogenation of organic compounds has been proposed in U.S. Pat. No. 4,287,365, and a similar iron doped Raney nickel catalyst has been used for the selective hydrogenation of halo nitro aromatic compounds in U.S. Pat. No. 5,801,284. Iron doped Raney nickel catalysts have also been proposed for hydrogenation of numerous organic functionalities in U.S. Pat. No. 6,368,996 and U.S. Pat. No. 6,395,934. In all the above patents the iron catalyst is used as a metal in a heterogeneous form, and as an additive to the well known Raney nickel. U.S. Pat. No. 5,126,485 claims the selective hydrogenation of halo nitro compounds in the presence of heterogeneous raney nickel, cobalt and iron catalysts.

Iron in combination with iridium, supported on carbon has been used as a catalyst for the hydrogenation of nitro compounds in U.S. Pat. No. 6,316,381. Here the main catalyst is the iridium with iron as an additive.

Iron hexacyano cobaltate complex and hexacyanoferrate compounds have been claimed for the hydrogenation of organic compounds including nitro compounds in U.S. Pat. No. 4,503,249 and U.S. Pat. No. 4,401,640. Here the metal complexes are supported on any suitable support like alumina etc and used in a heterogeneous form.

A combination of iron and a noble metal catalyst selected from palladium, platinum, and rhodium, supported on a suitable support is active for hydrogenation of dinitro aromatics and substituted dinitro aromatic compounds as mentioned in U.S. Pat. No. 5,105,012. A similar hydrogenation is feasible using iron in combination with platinum for selective hydrogenation as claimed in U.S. Pat. No. 4,212,824.

The use of iron carbonyl complexes in stoichiometric quantity for the reduction of nitrobenzene to aniline has been discussed by Vancheesan et al [J. Mol. Catal 1989, 52(2), 297-300], however the reaction mentioned therein is undertaken in the absence of hydrogen and the iron compound is employed in stoichiometric quantities and not used as a catalyst.

In all the examples mentioned above iron has never been used as a unique component of the catalyst, and also not in the soluble form. The only report where soluble iron compound is used pertains to its use for stoichiometric hydrogenation conducted in the absence of hydrogen.

Need therefore continues to exist for the catalytic equivalent of the stoichiometric nitro-reduction processes with high chemoselectivity. It is further desired that the needed catalyst should compliment catalytic performance with exclusive selectivity towards nitro group and physically the nature of the catalyst system should be such that it is easily recoverable preferably by simple phase separation. Thus, in view of the many possible applications involving the selective hydrogenation of nitro group to corresponding amines a new catalyst is a definite need.

### OBJECT OF THE INVENTION

The main object of the present invention is to provide an improved process for the selective hydrogenation of nitro aromatics, which obviates the draw backs associated with existing catalytic processes regarding chemoselectivity and ease of catalyst product separation and recycle.

Still another object of the present invention is to develop a catalytic process for the selective catalytic conversion of nitro functional group into amine in presence of other reducible functional groups such as aldehyde, ketone, olefin, nitrile etc

Yet another object of this invention is to use a soluble catalyst that can be operated in homogeneous or in biphasic conditions. An advantage of the biphasic mode of operation is the facile recovery and recycle of the catalyst system

### SUMMARY OF THE INVENTION

Accordingly, the present invention provides an improved process for the selective hydrogenation of nitro aromatics using homogeneous iron catalysts, wherein said catalyst is operated such that the catalyst is restricted to the one of the liquid phase existing in the reaction mixture while the starting materials and products predominantly exist in the other phase thereby facilitating recovery of the catalyst.

### DETAILED DESCRIPTION OF THE INVENTION

In order to achieve the above objectives, the present invention provides an improved process for selective hydrogenation of nitrogen containing aromatics, said process comprising the steps:
(a) mixing the nitrogen containing aromatics with a soluble iron compound/complex as a catalyst, optionally in presence of a solvent and a co-solvent;
(b) contacting the mixture of step (a) with gaseous hydrogen at a temperature ranging between 250 to 250° C. under hydrogen pressure in the range of 15 to 2000 psi for a time period greater than 2 hours to obtain a hydrogenated product, and
(c) separating the hydrogenated product thus obtained from the soluble iron catalyst.

In an embodiment of the present invention, wherein the nitrogen containing aromatics are selected from substituted or unsubstituted nitroaromatics, nitrosoaromatics and/or aromatic hydroxylamines.

In another embodiment of the present invention, wherein substituted or unsubstituted nitroaromatics, nitrosoaromatics and/or aromatic hydroxylamines have general formula Ar—Y, wherein Y is NO₂, NO or NHOH and Ar is an aromatic ring fragment selected from aryl fragments with or without one or more substitutions selected from the group consisting of C1 to C8 alkyl group, C1 to C4 aliphatic or aromatic vinyl group, C1 to C4 aliphatic or aromatic vinyloxy group, C1 to C8 alkoxy group, C₆H₅ to C₁₀H₈ aryloxy, fluro group, chloro group, bromo group, iodo group, hydroxy group, OCOalkyl group, OCOaryl group, COOH group, OH group, SH group, CN group, SO₃⁻ group, SO₂alkyl group, NH₂ group, Nhalkyl group, SO₂NH₂ group, SO₂N(alkyl)₂ group, SO₂Nhalkyl group, C1 to C4 aliphatic or aromatic aldehyde group, aliphatic or aromatic ketone group, C1 to C6 imino group, C1 to C6 ether group, thioester and sulfide.

In yet another embodiment of the present invention, wherein the catalyst used contains one or more soluble iron salts or one or more soluble iron organometallic complexes or mixture of one or more soluble iron salts and one or more soluble iron organometallic complexes.

In still another embodiment of the present invention, wherein the catalyst used is a soluble iron compound selected from iron salts of halogen acids, salts of oxo acids, iron organometallic complexes consisting of iron and a cordinating additive, wherein oxidation state of the iron atom ranges from 0 to 3.

In one more embodiment of the present invention, wherein the coordinating additives used is selected from the family of phosphines, bipyridyines, phenanthrolines, pentanediones and secondary or tertiary amines.

In one another embodiment of the present invention, wherein the coordinating additives used is selected from salts of water soluble derivatives of phosphines, bipyridyines, pentanediones, secondary or tertiary amine and ethylene diamine tetracetic acid.

In a further embodiment of the present invention, wherein the coordinating additive to iron ratio is in the range of 1:1 to 1:10.

In a further more embodiment of the present invention, wherein the catalyst is soluble in the reaction medium prior to commencement of the reaction or is solubilized under reaction conditions.

In an embodiment of the present invention, wherein catalyst is a unique iron compound or is formed as a mixture of entities containing as a constituent such that the entire mixture is soluble in intended liquid media prior to or during reaction conditions.

In another embodiment of the present invention, wherein the catalyst is soluble in organic medium or in aqueous medium and operates in homogeneous conditions or in biphasic conditions.

In yet another embodiment of the present invention, wherein when the catalyst operates in homogeneous conditions, the catalyst and the nitroaromatics, nitrosoaromatics and/or aromatic hydroxylamines are simultaneously soluble in organic medium or aqueous medium.

In still another embodiment of the present invention, wherein when the catalyst operates in biphasic conditions, the catalyst is soluble in organic medium and the nitroaromatics, nitrosoaromatics and/or aromatic hydroxylamines are soluble in aqueous medium or vice versa.

In one more embodiment of the present invention, wherein under biphasic operating conditions, the nitroaromatics, nitrosoaromatics and/or aromatic hydroxylamines are soluble in aqueous medium and the catalyst is soluble in organic medium.

In one another embodiment of the present invention, wherein the solvent used is selected from the group consisting of petroleum liquids such as crude oils, condensate and natural gases, aromatic liquids, alcohols, aliphatic or aromatic ethers, aliphatic or aromatic esters, nitrites, aliphatic or aromatic ketones, water or combinations thereof, wherein the solvent serves as a medium for dilution or facilitation of processing or separation.

In a further embodiment of the present invention, wherein the co-solvent used is selected from the group consisting of ethanol, acetone and acetonitrile, while facilitate the solubilization of the reactants into the catalyst phase.

In a further more embodiment of the present invention, wherein the concentration of the soluble iron catalyst is in the range of 10⁻⁴ to 10 mole % of the nitroaromatics, nitrosoaromatics and/or aromatic hydroxylamines.

In yet another embodiment of the present invention, the catalyst is selected from FeSO₄.7H₂O, Fe(NO₃)₃.9H₂O, Fe^{II}(acac)₃ or Fe^{II}/EDTANa₂.

In a feature of the of the present invention is also possible to carry out hydrogenation reaction with aqueous soluble nitroaromatic substrate such that catalyst is dissolved in organic medium while substrate and products can exist in aqueous phase.

In another feature the substituted and unsubstituted nitro aromatics, nitrosoaromatics and aromatic hydroxylamines may exist as a suspended solid in the liquid phase containing catalyst. The resulting aromatic amine can either phase separate from the catalyst containing liquid phase or remain as a solution.

In another feature the present invention may be performed in a batch or a continuous mode, wherein the catalyst recovered from these processing can be recycled after correction of volume without requiring any further operation of catalyst regeneration.

In yet another feature in biphasic operations of the present invention the catalyst containing phase can be directly recycled for subsequent reaction.

In another feature the catalyst of the present invention may be prepared by any known method for complexation of coordinating additive with iron atom or prepared in situ by addition of catalyst components to the reaction mixture.

In another embodiment the coordinating additive to iron ratio may be in the range of 1:1 to 1:10.

The process of the present invention is described hereinbelow with reference to the examples which are illustrative only and should not be construed to limit the scope of the present invention in any manner whatsoever.

The catalyst is generally prepared in situ by addition of the iron compound to the solvent/solvents and substrate mixture, followed by the coordinating additive in the requisite molar ratio desired. The catalyst is formed in-situ under the reaction conditions. Alternately the complex may also be formed separately by interaction of the iron compound with the coordinating additive taken in excess of the stoichiometric requirement, in a solvent like ethanol or acetonitrile such that both the metal compound and the coordinating additive are dissolved. The mixture is refluxed for a period of 4 hours to yield the iron complex which is then filtered and separated.

### EXAMPLE-1

This example demonstrates the hydrogenation of nitrobenzene carried out using different iorn catalysts and different solvents as mentioned in table-1. The hydrogenation of nitro benzene was carried out using Nitrobenzene: 9.6×10⁻² mol, solvent: 9×10⁻⁵ m³, catalyst 7.2×10⁻⁵ mol. The reaction mixture was charged in the 300 ml Parr autoclave and air in the autoclave was displaced with nitrogen and subsequently with hydrogen. Contents of the autoclave were heated to 150° C. and pressurized with 400 psi of hydrogen. The reaction was followed by absorption of gas and analysis of liquid phase, In all cases conversion was complete and selectivity to aniline was found to exceed 98%. Except example no 6 where in absence of iron compound no conversion was observed Apart from aniline other compounds were found to be azoxybenzene, azobenzene and hydrazobenzene.

TABLE 1

| Reaction No. | Catalyst | Solvent | Time (mins) |
|---|---|---|---|
| 1 | FeSO₄ 7H₂O | Toluene | 256 |
| 2 | FeSO₄ 7H₂O | Methanol | 185 |
| 3 | FeSO₄ 7H₂O | Water | 185 |
| 4 | Fe(NO₃)₃9H₂O | Methanol | 554 |
| 5 | Fe^{III}(acac)₃ | Toluene | 280 |
| 6 | — | Toluene | 500 |

### EXAMPLE-2

The example illustrates the Biphasic hydrogenation of nitrobenzene using different catalysts as mentioned in table-2.

The hydrogenation of nitro benzene was carried out under following conditions: Nitrobenzene: 9.6×10⁻² mol, toluene: 5×10⁻⁵ m³, water 5×10⁻⁵ m³′ catalyst FeSO₄.7H₂O: 7.2×10⁻⁵ mol. The reaction mixture was charged in the 300 ml Parr autoclave and air in the autoclave was displaced with nitrogen and subsequently with hydrogen. Contents of the autoclave were heated to 150° C. and pressurized with 400 psi of hydrogen. The reaction was followed by absorption of gas and analysis of liquid phase. In all cases conversion was complete and no iron was detected in organic phase except for reaction no 7 where in absence ligand total iron was found in organic phase. In reaction no 10, hydrogenation was carried out with 50 ml (0.483 mols) nitrobenzene.

TABLE 2

| Reaction | | Metal to | Time | Conversion | Selectivity |
|---|---|---|---|---|---|
| No | Catalyst | ligand ratio | (hours) | % | to aniline % |
| 7 | FeSO₄ | 1:0 | 7.38 | 66.28 | 98.1 |
| 8 | Fe^{II}/EDTANa₂ | 1:1 | 7.75 | 99.96 | 98.1 |
| 9 | Fe^{II}/EDTANa₂ | 1:5 | 9.83 | 88.2 | 97.3 |
| 10 | Fe^{II}/EDTANa₂ | 1:5 | 13.3 | 99.9 | 97.4 |

### EXAMPLE-3

The example illustrates the recycle of the catalyst in hydrogenation of nitrobenzene as mentioned in Table 3.

General procedure for recycle of aqueous biphasic catalyst: The reaction mixture discharged from reaction 9 [Table 2] was phase separated to remove aqueous phase. The aqueous phase was transferred to reactor to which was added 10 ml nitro benzene and hydrogenation was carried out as described earlier. In subsequent runs this procedure was repeated.

TABLE 3

| Reaction No. | Catalyst | Time (mins) | Selectivity |
|---|---|---|---|
| 11 | Aqueous solution recycled from | 635 | 95.8 |
| | experiment no 9 | | |
| 12 | Aqueous solution recycled from | 640 | 96.1 |
| | experiment no 11 | | |
| 13 | Aqueous solution recycled from | 626 | 96 |
| | experiment no 12 | | |
| 14 | Aqueous solution recycled from | 620 | 97 |
| | experiment no 13 | | |
| 15 | Aqueous solution recycled from | 631 | 97.1 |
| | experiment no 14 | | |

### EXAMPLE-4

The following example illustrates toe hydrogenation of various nitroaromatics as mentioned in Table 4.

The hydrogenation of exemplary nitroaromatics was conducted under the following conditions. The procedure was similar to that for biphasic reactions [reaction numbers 7-10, Table 2] Reaction conditions: Pressure: 400 psi; Temperature: 423K; Aqueous phase: 9×10⁻⁵ m³; Organic phase consists of neat substrate; catalyst FeSO₄.7H₂O: 7.2×10⁻⁵ mol; Fe: EDTANa₂ 1:5; conversion in all cases was complete; TOF calculated as mols of nitro compound converted per mol of Fe, § based on GC analysis.

TABLE 4

| | | | Yield of | |
|---|---|---|---|---|
| Reaction | | Substrate | corresponding | TOF |
| No | Substrate | (mols) | amine % | hr⁻¹ |
| 16 | Nitrobenzene | 0.090 | 98.5 | 529 |
| 17 | 2-nitrotoluene | 0.0849 | 98.75 | 457 |
| 18 | 2,4-dinitrotoluene | 0.056 | 84.63* | 134 |
| 19 | 4-nitrochlorobenzene | 0.0635 | 96.2 | 434 |
| 20 | 3-nitrochlorobenzene | 0.0635 | 99 | 400 |
| 21 | 2-nitrotoluene | 0.0849 | 98.75 | 462 |
| 22 | 2-nitroanisole | 0.0820 | 89.19 | 450 |
| 23 | 2-nitroaniline | 0.0725 | 98.2 | 116 |
| 24 | 4-nitrobenzoic acid | 0.0598 | 85.8 | 393 |
| 25 | 4-nitro acetophenone | 0.06 | 99 | 208 |
| 26 | 4-nitro benzylnitrile | 0.061 | 90 | 99 |

### ADVANTAGES OF THE PRESENT INVENTION

The present invention provides an improved catalyst and process for the selective hydrogenation of substituted nitro aromatics, nitrosoaromatics and aromatic hydroxyl amines with gaseous hydrogen to the corresponding amines in the presence of soluble iron compound as a catalyst. The present invention uses a soluble iron compound or complex as a catalyst, which is not disclosed in any prior art and is a new discovery. The present invention provides a highly chemoselective route for the manufacture of substituted aromatic amines using a homogeneous catalyst. The catalysts in the prior art are normally heterogeneous supported metal catalysts wherein iron is used as an additive, and are generally less selective hydrogenation catalysts. The use of two phase solvent systems has manifold advantages pertaining to catalyst product separation, catalyst recycle and recovery, and prevention of deactivation of catalyst by interaction with products or byproducts. In the present invention the product being more soluble in the non catalyst phase is swept out leaving no possibility of interaction of catalyst and product thereby decreasing the risk of deactivation of catalyst. Additionally the present invention being applied in a solution form the advantages of temperature control and processing are evident.

1. An improved process for selective hydrogenation of nitrogen containing aromatics, said process comprising the steps:
(a) mixing the nitrogen containing aromatics with a soluble iron compound/complex as a catalyst, optionally in presence of a solvent and a co-solvent;
(b) contacting the mixture of step (a) with gaseous hydrogen at a temperature ranging between 25° to 250° C. under hydrogen pressure in the range of 15 to 2000 psi for a time period greater than 2 hours to obtain a hydrogenated product, and
(c) separating the hydrogenated product thus obtained from the soluble iron catalyst.

2. A process as claimed in claim 1, wherein the nitrogen containing aromatics are selected from substituted or unsubstituted nitroaromatics, nitrosoaromatics and/or aromatic hydroxylamines.

3. A process as claimed in claim 2, wherein substituted or unsubstituted nitroaromatics, nitrosoaromatics and/or aromatic hydroxylamines have general formula Ar—Y, wherein Y is NO₂, NO or NHOH and Ar is an aromatic ring fragment selected from aryl fragments with or without one or more substitutions selected from the group consisting of C1 to C8 alkyl group, C1 to C4 aliphatic or aromatic vinyl group, C1 to C4 aliphatic or aromatic vinyloxy group, C1 to C8 alkoxy group, C₆H₅ to C₁₀H₈ aryloxy, fluro group, chloro group, bromo group, iodo group, hydroxy group, OCOalkyl group, OCOaryl group, COOH group, OH group, SH group, CN group, SO₃⁻ group, SO₂alkyl group, NH₂ group, Nhalkyl group, SO₂NH₂ group, SO₂N(alkyl)₂ group, SO₂Nhalkyl group, C1 to C4 aliphatic or aromatic aldehyde group, aliphatic or aromatic ketone group, C1 to C6 imino group, C1 to C6 ether group, thioester and sulfide.

4. A process as claimed in claim 1, wherein the catalyst used contains one or more soluble iron salts or one or more soluble iron organometallic complexes or mixture of one or more soluble iron salts and one or more soluble iron organometallic complexes.

5. A process as claimed in claim 1, wherein the catalyst used is a soluble iron compound selected from iron salts of halogen acids, salts of oxo acids, iron organometallic complexes consisting of iron and a cordinating additive, wherein oxidation state of the iron atom ranges from 0 to 3.

6. A process as claimed in claim 5, wherein the coordinating additives used is selected from the family of phosphines, bipyridyines, phenanthrolines, pentanediones and secondary or tertiary amines.

7. A process as claimed in claim 5, wherein the coordinating additives used is selected from salts of water soluble derivatives of phosphines, bipyridyines, pentanediones, secondary or tertiary amine and ethylene diamine tetracetic acid.

8. A process as claimed in claim 5, wherein the coordinating additive to iron ratio is in the range of 1:1 to 1:10.

9. A process as claimed in claim 1, wherein the catalyst is soluble in the reaction medium prior to commencement of the reaction or is solubilized under reaction conditions.

10. A process as claimed in claim 1, wherein catalyst is a unique iron compound or is formed as a mixture of entities containing as a constituent such that the entire mixture is soluble in intended liquid media prior to or during reaction conditions.

11. A process as claimed in claim 1, wherein the catalyst is soluble in organic medium or in aqueous medium and operates in homogeneous conditions or in biphasic conditions.

12. A process as claimed in claim 10, wherein when the catalyst operates in homogeneous conditions, the catalyst and the nitroaromatics, nitrosoaromatics and/or aromatic hydroxylamines are simultaneously soluble in organic medium or aqueous medium.

13. A process as claimed in claim 10, wherein when the catalyst operates in biphasic conditions, the catalyst is soluble in organic medium and the nitroaromatics, nitrosoaromatics and/or aromatic hydroxylamines are soluble in aqueous medium or vice versa.

14. A process as claimed in claim 12 wherein under biphasic operating conditions, the nitroaromatics, nitrosoaromatics and/or aromatic hydroxylamines are soluble in aqueous medium and the catalyst is soluble in organic medium.

15. A process as claimed in claim 1, wherein the solvent used is selected from the group consisting of petroleum liquids such as crude oils, condensate and natural gases, aromatic liquids, alcohols, aliphatic or aromatic ethers, aliphatic or aromatic esters, nitrites, aliphatic or aromatic ketones, water or combinations thereof, wherein the solvent serves as a medium for dilution or facilitation of processing or separation.

16. A process as claimed in claim 1, wherein the co-solvent used is selected from the group consisting of ethanol, acetone and acetonitrile, while facilitate the solubilization of the reactants into the catalyst phase.

17. A process as claimed in claim 1, wherein the concentration of the soluble iron catalyst is in the range of 10⁻⁴ to 10 mole % of the nitroaromatics, nitrosoaromatics and/or aromatic hydroxylamines.

18. A process as claimed in claim 1, wherein the catalyst is selected from FeSO₄.7H₂O, Fe(NO₃)₃.9H₂O, Fe^{II}(acac)₃ or Fe^{II}/EDTANa₂.
